(19) United States
(12) Patent Application Publication
(10) Pub. No.: US 2016/0129275 A1
(43) Pub. Date: May 12, 2016

(54) MAGNETIC HYPERTHERMAL ABLATION OF TUMOR CELLS

(71) Applicant: Henry Keith Vickery, Greenbrier, TN (US)

(72) Inventor: Henry Keith Vickery, Greenbrier, TN (US)

(21) Appl. No.: 14/925,371

(22) Filed: Oct. 28, 2015

Related U.S. Application Data
(60) Provisional application No. 62/076,296, filed on Nov. 6, 2014.

(51) Int. Cl. A61N 2/00; A61N 2/02; A61K 41/00

(52) U.S. Cl. CPC A61N 2/004; A61K 41/0052; A61N 2/02

(57) ABSTRACT

A method of treating cancer cells in a tumor comprising: a) administering a pharmaceutical composition comprising: i) functionalized magnetic gold nanoparticles (GNP) coated with a polymer giving them a positive charge and ii) a pharmaceutically acceptable carrier therefore; and b) exposing the tumor to an intense and rapidly fluctuating magnetic field sufficient to induce eddy currents capable of hyperthermally ablating the cancer cells.

### CROSS-REFERENCE TO RELATED APPLICATION

This application claims the benefit of priority of U.S. provisional application No. 62/076,296, filed Nov. 6, 2014, the entire contents and disclosure of which are herein incorporated by reference.

### BACKGROUND OF THE INVENTION

Conventional tumor treatment protocols are often dangerous in that, although they are designed to kill cancer cells, they do not satisfactorily distinguish between healthy cells and those that are malignant, and very often result in the destruction of the healthy cells as well as cancer cells. Chemo- and radiation-therapies are well known to (1) destroy healthy tissues along with the cancerous tissues which can lead to death; (2) produce life threatening side effects; (3) produce illness or sick feelings; and (4) be painful.

Thus, there exists a great need for cancer treatments that target only cancer cells and do not adversely affect healthy tissue.

### SUMMARY OF THE INVENTION

One embodiment of the invention relates to a method of treating an individual afflicted with cancer cells, the method comprising: a) administering to the individual a pharmaceutical composition comprising: i) functionalized gold nanoparticles (GNP) coated with a polymer giving them a positive charge and ii) a pharmaceutically acceptable carrier therefore; and b) exposing the tumor to an intense and rapidly fluctuating magnetic field sufficient to induce eddy currents capable of hyperthermally ablating the cancer cells.

### BRIEF DESCRIPTION OF THE DRAWINGS

FIG. 1 is a block diagram/flow chart of the present invention;

FIG. 2 is a perspective view of apparatus employed in the practice of the present invention;

FIG. 3 is an exploded view of apparatus employed in the practice of the present invention;

FIG. 4 is a right side elevation view of apparatus employed in the practice of the present invention; and

FIG. 5 is a rear elevation view of apparatus employed in the practice of the invention.

### DETAILED DESCRIPTION OF THE INVENTION

The following detailed description is of the best currently contemplated mode for carrying out exemplary embodiments of the invention. The description is not to be taken in a limiting sense, but is made merely for the purpose of illustrating the general principles of the invention, since the scope of the invention is best defined by the appended claims.

The present invention is predicated on the discoveries (1) that gold nanoparticles can be functionalized; i.e., made to have a greater affinity for tumor cells than healthy cells by coating them with a polymer that imparts a positive charge thereto, and (2) that cancerous tumor cells associated with the functionalized gold particles can be destroyed without the concomitant destruction of surrounding healthy cells when they are exposed to an intense and rapidly fluctuating magnetic field sufficient to induce eddy currents capable of hyperthermally ablating the cancer cells. Following the treatment, the body effectively removes the destroyed tissue utilizing the body's own enzymes and waste disposal process.

The procedure does not: (1) destroy healthy tissues; (2) produce any known deleterious side effects; or (3) produce illnesses or painful side effects.

The system of the invention utilizes magnetically induced hyperthermia to destroy cancer cells without the destruction of healthy tissue.

The crux of the invention resides in the magnetic generation of eddy currents in the GNPs attached to the tumor cells, which raises the temperature of the tumor cells, thus killing them. Optimum results are achieved when utilizing a multi-turn magnetic coil, e.g., a typical Helmholtz coil. Depending upon the total volume and depth of the target tumor, typical operational frequencies lie between 1-30 MHz, with magnetic strengths greater than or equal to 500 Microteslas.

Once the gold heats the interior of the cell above 106-107 degrees F., the cell will lysis, as will the lyposomal sac which releases enzymes that catalyze the metabolism of the lysed cell residue. The gold is then free to be eliminated from the body through the urine.

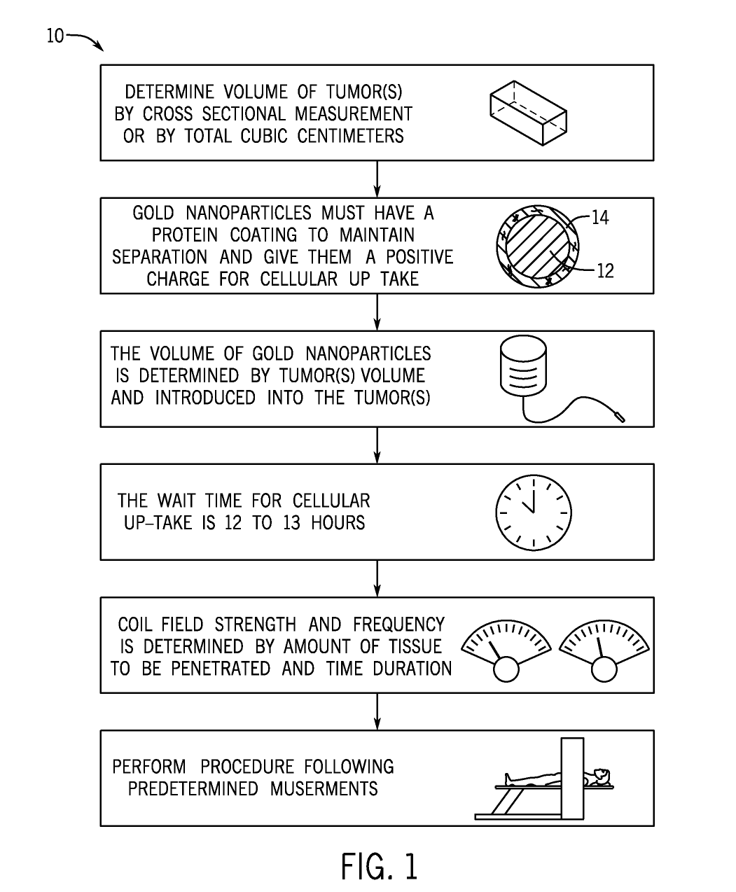

FIG. 1 is a flow chart 10 of the invention depicting the gold nanoparticles 12 coated with polymer layer 14. The polymer coated gold nanoparticles have a 600 times greater affinity for tumors cells than any other cells in the body. Any polymer that imparts a positive electrical charge to the gold nanoparticles may be employed in the practice of the invention. Exemplary, but not limitative of such polymers are the polyethylene glycol ethers, hexadimethrine bromide (Polybrene), dextran sulfate, polyethylenimine, and the like. A preferred such polymer is polyethylene glycol methyl ether: PEG 5000. However, it is to be understood that any polymer capable of positively charging the gold nanoparticles may be employed.

### Example

The treatment embodied by the invention was performed on a 45 year old Caucasian female patient G2P2 who had experienced abnormal merometrorregia for approximately 4 months. Colposcopy revealed invasive moderately differenced squamous cell cancer. After radical hysterectomy and CT, PET scan the cancer was staged at T2A2 M. PET/CT scans s/p radical hysterectomy left pelvic, retroperitoneal and rettrocrural lymph nodes involvement and small thoracic retroaortic lymph modes adjacent to the distal descending thoracic aorta. The Patient initiated a 6 week course of cis-platin and radiation of pelvis, but 3 weeks in was unable to tolerate side effects.

The patient was administered an allergy exam and observed for 30 minutes following the test for adverse reactions. BP was 125/88, HR 89, R 18, O2 SAT 96%.

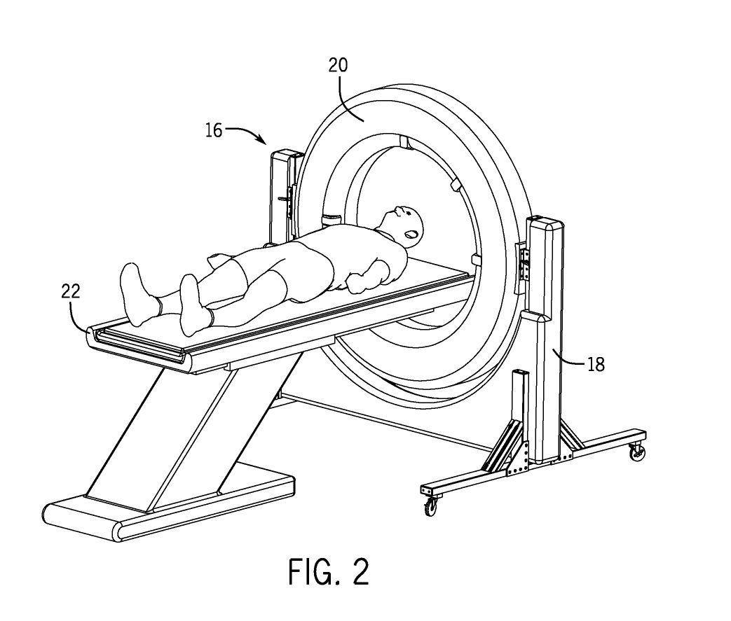

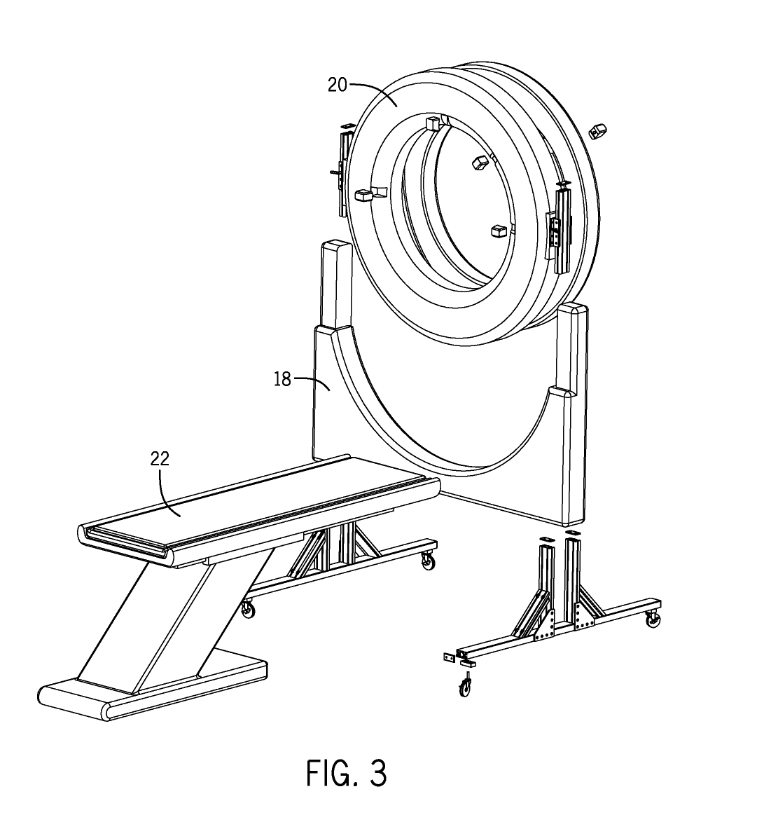

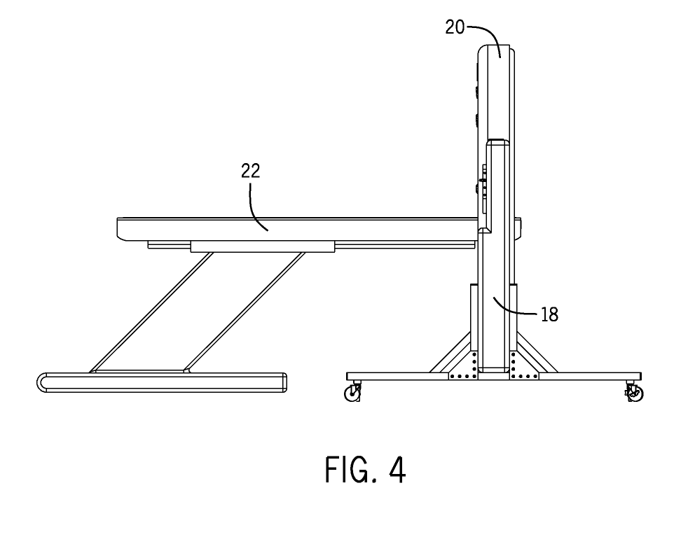

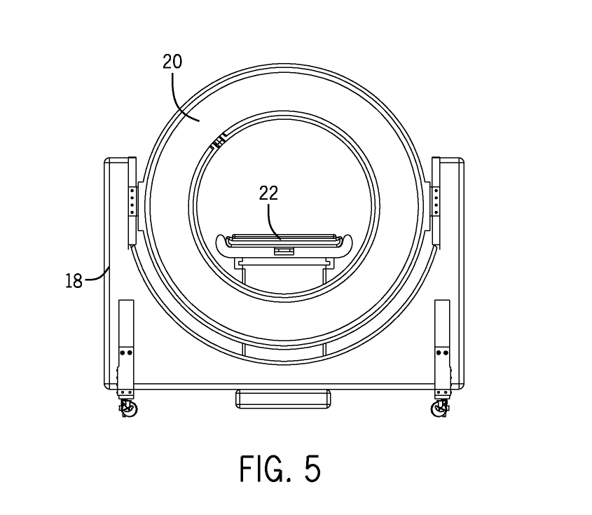

Patient underwent treatment for cervical, lymph, esophageal cancers with IV of nanoparticle gold surface coated with PEG-5000 polymer. Patient received 70 mg of coated gold nanoparticles and 14 hours later was placed in the Helmholtz coil 20 of the hyperthermic device 16, comprising a frame 18 and a patient table 22, depicted in FIGS. 2-5, wherein she was subjected to an electromagnetic field starting at the lower pelvis and upon being moved 6 inches up every two hours at a time from pelvis to chin as follows:

1. Left hip lymph. Exposure to a high intensity rapidly varying magnetic field for 2 hours. Tolerated procedure without incident. Patient took approximately 15 min. break before resuming.

2. Vaginal. Exposure to a high intensity rapidly varying magnetic field for 2 hours. Tolerated procedure without incident. Patient took approximately 10 min. break before resuming.

3. Lower lumbar lymph. Exposure to a high intensity rapidly varying magnetic waves for 2 hours. Tolerated procedure without incident.

4. Middle back lymph. Exposure to a high intensity rapidly varying magnetic field for 2 hours. Tolerated procedure without incident. Patient took approximately 15 min. break before resuming.

5. Upper back lymph. Exposure to a high intensity rapidly varying magnetic field for 2 hours. Tolerated procedure without incident. Patient took approximately 15 min. break before resuming.

6. Esophageal and upper neck. Exposure to a high intensity rapidly varying magnetic field for 2 hours. Tolerated procedure without incident. Patient took approximately 15 min. break. Procedure ended.

The patient tolerated all procedures without incident. Post procedure: VS: BP 133/89, HR 87, R 18, O2 SAT 98%.

Within 3 days the left groin pain that had been attributed to cancer pain completely resolved and a PET/CT scan performed 5 weeks later showed complete resolution of the cancer.

It should be understood, of course, that the foregoing relates to exemplary embodiments of the invention and that modifications may be made without departing from the spirit and scope of the invention as set forth in the following claims.

What is claimed is:

1. A method of treating an individual afflicted with cancer cells; the method comprising:
a) administering to the individual an effective amount of a pharmaceutical composition comprising:
i) magnetic gold nanoparticles coated with a functionalizing amount of a polymer that imparts a positive charge thereto and
ii) a pharmaceutically acceptable carrier therefore, and
b) exposing the tumor to an intense and rapidly fluctuating magnetic field sufficient to induce eddy currents capable of hyperthermally ablating the cancer cells.

2. The method of claim 1, wherein said magnetic field is 1-30 MHz having a strength greater than or equal to 500 microteslas.

3. The method of claim 1, wherein said positively charged polymer is a polyethylene glycol ether, hexadimethrine bromide (Polybrene), dextran sulfate, or polyethylenimine.

4. The method of claim 1, wherein said positively charged polymer is a polyethylene glycol ether.

5. The method of claim 4, wherein said positively charged polymer is PEG 5000.

6. The method of claim 1, wherein said composition is administered intravenously.
